SEQUENCE LISTING (31 sequences)

SEQ ID NO: 1 - protein, 5 aa, Artificial
  synthetic peptide
FVFLM

SEQ ID NO: 2 - protein, 5 aa, Artificial
  synthetic peptide
FVFAM

SEQ ID NO: 3 - protein, 5 aa, Artificial
  synthetic peptide
FVALM

SEQ ID NO: 4 - protein, 5 aa, Artificial
  synthetic peptide
FVFLA

SEQ ID NO: 5 - protein, 5 aa, Artificial
  synthetic peptide
FLVFI

SEQ ID NO: 6 - protein, 5 aa, Artificial
  synthetic peptide
FLMII

SEQ ID NO: 7 - protein, 5 aa, Artificial
  synthetic peptide
FLFVL

SEQ ID NO: 8 - protein, 5 aa, Artificial
  synthetic peptide
FLFVV

SEQ ID NO: 9 - protein, 5 aa, Artificial
  synthetic peptide
FLFLI

SEQ ID NO: 10 - protein, 5 aa, Artificial
  synthetic peptide
FLFFI

SEQ ID NO: 11 - protein, 5 aa, Artificial
  synthetic peptide
FLMFI

SEQ ID NO: 12 - protein, 5 aa, Artificial
  synthetic peptide
FMLLI

SEQ ID NO: 13 - protein, 5 aa, Artificial
  synthetic peptide
FIIMI

SEQ ID NO: 14 - protein, 5 aa, Artificial
  synthetic peptide
FLFCI

SEQ ID NO: 15 - protein, 5 aa, Artificial
  synthetic peptide
FLFAV

SEQ ID NO: 16 - protein, 5 aa, Artificial
  synthetic peptide
FVYLI

SEQ ID NO: 17 - protein, 5 aa, Artificial
  synthetic peptide
FAFLM

SEQ ID NO: 18 - protein, 5 aa, Artificial
  synthetic peptide
AVFLM

SEQ ID NO: 19 - protein, 10 aa, Homo sapiens
GITKVFSNGA

SEQ ID NO: 20 - protein, 10 aa, Homo sapiens
DLSGVTEEAP

SEQ ID NO: 21 - protein, 10 aa, Homo sapiens
LKLSKAVHKA

SEQ ID NO: 22 - protein, 10 aa, Homo sapiens
VLTIDEKGTE

SEQ ID NO: 23 - protein, 10 aa, Homo sapiens
AAGAMFLEAI

SEQ ID NO: 24 - protein, 10 aa, Homo sapiens
PMSIPPEVKF

SEQ ID NO: 25 - protein, 10 aa, Homo sapiens
NKPFVFLMIE

SEQ ID NO: 26 - protein, 10 aa, Homo sapiens
QNTKSPLFMG

SEQ ID NO: 27 - protein, 8 aa, Homo sapiens
KVVNPTQK

SEQ ID NO: 28 - protein, 417 aa, Homo sapiens
MPSSVSWGILLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 29 - protein, 22 aa, Homo sapiens
LEAIPMSIPPEVKFNKPFVFLM

SEQ ID NO: 30 - protein, 20 aa, Homo sapiens
LEAIPMSIPPEVKFNKPFVF

SEQ ID NO: 31 - protein, 80 aa, Homo sapiens
GADLSGVTEEAPLKLSKAVHKAVLTIDEKGTEAAGAMFLERIPVSIPPEVKFNKPFVFLMIEQNTKSPLFMGKVVNPTQK

(19) United States
(12) Patent Application Publication
(10) Pub. No.: US 2012/0225813 A1
(43) Pub. Date: Sep. 6, 2012

(54) COMPOSITIONS, METHODS AND USES FOR TREATMENT OF TYPE 1 DIABETES

(76) Inventors: Eli C. LEWIS, Be'er Sheva (IL); Peter GOTTLIEB, Englewood, CO (US); Charles A. DINARELLO, Boulder, CO (US); Leland SHAPIRO, Denver, CO (US); Aaron MICHELS, Aurora, CO (US)

(21) Appl. No.: 13/159,200

(22) Filed: Jun. 13, 2011

Related U.S. Application Data
(60) Provisional application No. 61/354,069, filed on Jun. 11, 2010.

(51) Int. Cl. A61K 38/57; A61P 3/10

(52) U.S. Cl. 514/7.3

(57) ABSTRACT

Embodiments herein concern compositions and methods for treating diabetes in a subject.

### PRIORITY

The instant application claims the benefit under 35 USC §119 (e) of U.S. Provisional Patent Application Ser. No. 61/354,069 filed Jun. 11, 2010. This provisional application is incorporated herein by reference in its entirety for all purposes.

### FIELD OF USE

Embodiments herein report methods, compositions and uses for treatment of a subject having Type 1 diabetes. In some embodiments, the subject having Type 1 diabetes may have detectible c-peptide levels and/or detectible insulin production and/or residual islet cell function. In certain embodiments, compositions and methods herein concern treating a subject having Type 1 diabetes for a short duration.

### BACKGROUND

There are two types of diabetes, Type 1 and Type 2. Type 1 diabetes (T1D) is the immune mediated form of diabetes. It is an autoimmune disease that is organ specific for the pancreatic beta cells. The disease pathogenesis involves T-cell infiltration into the islets of the pancreas that subsequently destroys the insulin producing cells, and result in overt symptoms of disease. Currently there is no known cure for TID and treatment for the disease consists of lifelong administration of insulin. Despite treatment with insulin therapy long-term complications, including nephropathy, retinopathy, neuropathy, and cardiovascular disease, can result.

Currently, there are no treatments available for preservation of insulin production outside of research studies. Previously, the combination of cyclosporine and azothioprine plus glucocorticoids was studied and found to have unfavorable side effect profiles. Studies using anti-CD3 monoclonal antibodies have demonstrated the ability to slow the beta cell destruction in recent onset T1D. Again, the anti-CD3 studies also had serious adverse events and induce immunosuppression. Several antigen studies are also under way. Previously, antigen therapy with oral insulin demonstrated a delay in onset of T1D in subjects at increased risk for developing T1D. Several other antigens are also being studied in new onset T1D including proinsulin DNA vaccines and GAD65 vaccines.

### SUMMARY

Embodiments herein report methods, compositions and uses for treatment of diabetes in a subject. In some embodiments, a subject can be a subject having Type 1 diabetes (T1D). In accordance with these embodiments, Type-1 diabetics can be subjects having been diagnosed in a certain time period having remaining insulin production. In these examples, insulin production can be about 1 to about 10 pmols/ml or about 2 to about 5 pmols/ml insulin production by the subject. In other embodiments, a subject may be treated with compositions disclosed herein to modulate destruction of islet cell function. In certain embodiments, modulation of islet cells can include induction of function and/or recovery of islet cell populations in the subject. In other embodiments, compositions and methods disclosed herein may be used to treat Type 1 diabetics for about 5 weeks to about 2 years, or about 8 weeks to about 1 year etc. with compositions disclosed herein.

In other embodiments, compositions and methods disclosed herein can include periodic AAT treatment of a subject in order to reduce islet cell destruction of a subject contemplated herein. Thus, treatment may be for a short duration compared to long term treatment using insulin replacement therapy. Depending on the situation of the patient, periodic long term administration of compositions disclosed herein may be needed.

### BRIEF DESCRIPTION OF THE DRAWING

FIG. 1 illustrates short term AAT treatment of diabetic NOD mice.

### DESCRIPTION OF ILLUSTRATIVE EMBODIMENTS

### Definitions

As used herein, “a” or “an” may mean one or more than one of an item.

As used herein, “about” can mean plus or minus 10%, for example, about 10 minutes can mean from 9 to 11 minutes.

As used herein “immunomodulatory drugs or agents”, can mean, e.g., agents capable of acting on the immune system, directly or indirectly, e.g., by stimulating or suppressing a cellular activity of a cell in the immune system, e.g., T-cells, B-cells, macrophages, or antigen presenting cells (APC, dendritic cells), or by acting upon components outside the immune system which, in turn, stimulate, suppress, or modulate the immune system, e.g. cytokines, hormones, receptor agonists or antagonists, and neurotransmitters; immunomodulators (e.g., immunosuppressants or immunostimulants).

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times, temperature and other details may be modified through routine experimentation. In some cases, well known methods or components have not been included in the description.

Type 1 diabetes is an immune-mediated form of diabetes. It is an autoimmune disease that is organ specific for the pancreatic beta cells. The disease pathogenesis involves T-cell infiltration into the islets of the pancreas that subsequently destroys the insulin producing cells, and result in overt symptoms of disease. While the progress to complete insulin dependence can occur quickly after clinical onset, initially after diagnosis the pancreas is able to produce a significant amount of insulin (the “honeymoon” period). During this state, a subject will have less glucose variability. The Diabetes Control and Complications Trial (DCCT) found that 20% of patients studied, who were within 5 years of diagnosis, had remaining insulin production (2-5 pmol/ml). At this time immunologic intervention can potentially save beta cell function and reduce reliance on insulin. With the increasing incidence of TID, therapies aimed at altering the underlying autoimmune process need to be investigated. Even partial beta cell function is beneficial as patients that maintain endogenous insulin production have better metabolic control than those who rely solely on exogenous insulin and improved metabolic control reduces the long-term complications from diabetes. Therapies that halt beta cell destruction can lead to continued endogenous insulin production, greatly improving metabolic control and reducing complications in TID.

In certain embodiments, the subject or mammal is a human.

In other embodiments, the subject or mammal can be a domesticated or a non-domesticated mammal.

In some embodiments, a subject having been recently diagnosed with Type 1 diabetes (T1D) having residual beta cell function may be treated with alpha 1 antitrypsin (AAT) using regimens disclosed herein. In certain embodiments, the subject is a human. In other embodiments, the human is a juvenile. In some embodiments, the human is an adult.

AAT is a serine protease inhibitor that has been used safely since 1987 in humans. It is known that AAT deficiency can lead to emphysema and liver cirrhosis in humans. AAT has been shown to have anti-inflammatory effects and potentiate immune tolerance. In certain embodiments, AAT-associated molecules used in the methods and compositions herein can include, but are not limited to, compositions of naturally occurring AAT (394 AA length molecule making up approximately 90% of AAT isolated from serum). These compositions may be partially or fully purified compositions of AAT molecules.

In other embodiments, α1-antitrypsin used in the methods and compositions herein can be a commercially available composition and can include, but is not limited to, Aralast™ (Baxter), Zemaira™ (Aventis Behring), Prolastin™ (Talecris, N.C.), Prolastin C™ (Talecris, N.C.), Aerosolized AAT™ or Intravenous AAT™ (Kamada' Ltd. Israel) or any other commercially available composition or any combination thereof. In other embodiments, AAT used in methods and compositions herein can include naturally-occurring or a mutant form of AAT purified or partially purified from a mammalian source.

In some embodiments, pharmaceutical compositions contemplated herein are administered orally, systemically, via an implant, intravenously, topically, intrathecally, intracranially, intraventricularly, by inhalation or nasally. In certain embodiments, inhalable AAT may be manufactured by Kamada (Israel). In other embodiments, pharmaceutical compositions contemplated herein are administered intravenously by regimens disclosed herein and for certain periods of time described.

In certain embodiments, synthetic and/or naturally occurring peptides/proteins may be used in compositions and methods herein for example, providing other than serine protease inhibitor activity of AAT. Homologues, natural peptides, or peptidyl derivatives, e.g., aldehyde or ketone derivatives of such peptides are also contemplated herein. Without limiting to AAT, compounds like oxadiazole, thiadiazole and triazole peptoids and substances can include, but are not limited to, certain phenylenedialkanoate esters, CE-2072, UT-77, and triazole peptoids. Examples of analogues are TLCK (tosyl-L-lysine chloromethyl ketone) or TPCK (tosyl-L-phenylalanine chloromethyl ketone) or any combination thereof.

In certain embodiments, AAT could potentially inhibit the inflammation associated with TID and/or enhance immune tolerance resulting in sustained beta cell function. With its long term pharmacologic use along with the above data on inflammation in TID and the pre-clinical studies in the NOD mouse model, AAT is likely a safe and potentially beneficial therapy for example, for recent onset type 1 diabetics.

In one particular embodiment, the present inventions provide for methods for reducing levels and activities of cytokines such as TNFα (tumor necrosis factor alpha). For example, the composition can include alpha-1-antitrypsin or analog thereof or combination thereof alone or in combination with other therapies.

In one embodiment of the present invention a composition may include compounds that engage molecules that bind the SEC receptor for treating an early onset Type 1 diabetic subject. In each of the recited methods, an alpha-1 antitrypsin (e.g. mammalian derived) can include a series of peptides including carboxyterminal amino acid peptides corresponding to AAT. These pentapeptides can be represented by a series of peptides, several are equally acceptable including FVFLM (SEQ ID NO:1), FVFAM (SEQ ID NO:2), FVALM (SEQ ID NO:3), FVFLA (SEQ ID NO:4), FLVFI (SEQ ID NO:5), FLMII (SEQ ID NO:6), FLFVL (SEQ ID NO:7), FLFVV (SEQ ID NO:8), FLFLI (SEQ ID NO:9), FLFFI (SEQ ID NO:10), FLMFI (SEQ ID NO:11), FMLLI (SEQ ID NO:12), FIIMI (SEQ ID NO:13), FLFCI (SEQ ID NO:14), FLFAV (SEQ. ID NO:15), FVYLI (SEQ ID NO:16), FAFLM (17), AVFLM (SEQ ID NO:18), and any combination thereof.

In certain embodiments, AAT peptides contemplated for use in the compositions and methods of the present invention are also intended to include any and all of those specific AAT peptides of SEQ ID NO:31 (naturally-occurring AAT of 394 amino acids, the most common form is the M type with subtypes M1, M2, M3 etc. are also contemplated herein). All AAT polypeptides are contemplated of use in methods disclosed herein, that possess both anti-inflammatory activity and immune regulatory activity. Any combination of consecutive amino acids simulating AAT or AAT-like activity may be used, such as amino acids ranging from 315-394, amino acids ranging from 325-384, 340-380 etc. In addition, combinations of consecutive amino acid sequences such as 5-mers or 10-mers of the carboxy terminus can also be used. For example, any combinations of 5-mers or 10-mers from SEQ ID NO:30 AAs 314-394 can be used in compositions contemplated herein. Another example is provides for use of any AAT derived carboxyterminal peptide below.

As contemplated herein, the last amino acids in a protein include the carboxy terminus. In certain embodiments, the carboxyl domain of AAT going backwards from the carboxy terminus is defined as those amino acids most conserved among the difference species and do not participate in the protease binding domain of AAT. In addition, in other embodiments, AAT protease binding domain can be mutated in order to reduce or eliminate the protease function of the molecule and not inhibit elastase activity; these molecules can be used in any composition contemplated herein. In certain embodiments, a mutated AAT can be used to protect remaining islet cells in a T1D subject either supplied directly to the islets and/or administered to a subject in need of such a treatment. In other embodiments, a mutated molecule (e.g. having reduced or essentially no protease activity) retains its anti-inflammatory effects and/or immunomodulatory effects and can be used as an anti-inflammatory molecule in a subject having a diabetic condition. One skilled in the art would understand a non-protease binding domain of AAT as well as what is termed the carboxyterminal last 80 amino acids.

In each of the above-recited methods, al-antitrypsin or carboxyterminal peptide derivatives thereof are contemplated for use in a composition herein. These peptide derivatives may include but are not limited to amino acid peptides containing the last 80 carboxyterminal derived amino acids of AAT, GITKVFSNGA (SEQ ID NO:19), DLSGVTEEAP (SEQ ID NO:20), LKLSKAVHKA (SEQ ID NO:21), VLTIDEKGTE (SEQ ID NO:22), AAGAMFLEAI (SEQ ID NO:23), PMSIPPEVKF (SEQ ID NO:24), NKPFVFLMIE (SEQ ID NO:25), QNTKSPLFMG (SEQ ID NO:26), KVVNPTQK (SEQ ID NO:27), LEAIPMSIPPEVKFNKPFVFLM (SEQ ID NO:28); and LEAIPMSIPPEVKFNKPFVF (SEQ ID NO:29), GADLSGVTEEAPLKLSKAVHKAV LTIDEKGTEAAGAMFLERIPV SIPPEVKFNKPFVFLMIEQNTKSPLFMGKVVNPTQK (SEQ ID NO:30) or any combination thereof.

In accordance with embodiments of the present invention, the peptide can be protected or derivitized in by any means known in the art for example, N-terminal acylation, C-terminal amidation, cyclization, etc. In a specific embodiment, the N-terminus of the peptide is acetylated.

### Proteins

In certain embodiments, proteins may be purified or partially purified and used as compositions for methods disclosed herein. Proteins contemplated of use herein can include, but is not limited to, purified or partially purified compositions of AAT, for example, naturally-occurring forms of AAT or synthetically made compositions of AAT. In addition, mutant forms of AAT are contemplated (e.g. mutations at AA 358 or mutations that increase the half-life of AAT).

### Pharmaceutical Compositions:

Embodiments herein provide for administration of compositions to subjects in a biologically compatible form suitable for pharmaceutical administration in vivo. By “biologically compatible form suitable for administration in vivo” is meant a form of the active agent (e.g. pharmaceutical chemical, protein, gene, antibody, or anti-viral agent) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

In one embodiment, the compound (e.g. pharmaceutical chemical, protein, gene, antibody, or anti-viral agent) may be administered to a subject in need thereof subcutaneously, intravenously, by oral administration, inhalation, transdermally, intravaginally, topically, intranasally, rectally or a combination thereof. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be administered intravenously. In one particular embodiment, the compound may be administered intranasally, such as inhalation.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under some conditions of storage and use, these preparations may contain a preservative to prevent the growth of microorganisms.

Pharmaceutical compositions suitable for injectable use may be administered by means known in the art. For example, sterile aqueous solutions (where water soluble) or dispersions and sterile powders for the extemporaneous preparation of sterile injectable solutions or dispersion may be used. In all cases, the composition can be sterile and can be fluid to the extent that easy syringability exists. It might be stable under the conditions of manufacture and storage and may be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by heating, exposing the agent to detergent, irradiation or adding various antibacterial or antifungal agents.

Sterile injectable solutions can be prepared by incorporating active compound (e.g. a compound capable of inhibiting viral infection) in an amount determined to be appropriate by a healthcare provider in a solvent with one or a combination of ingredients enumerated above, followed, for example, by filter sterilization.

Aqueous compositions can include an effective amount of a therapeutic compound, peptide, epitopic core region, stimulator, inhibitor, and the like, dissolved or dispersed in a pharmaceutically acceptable carrier or aqueous medium. Compounds and biological materials disclosed herein can be purified by means known in the art.

Solutions of the active compounds as free-base or pharmacologically acceptable salts can be prepared and suitably mixed with for example, a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms. Prolonged absorption of the injectable or ingestible compositions can be brought about by compositions of agents delaying absorption, for example, aluminum monostearate, gelatin or the like. In other embodiments, a composition contemplated herein can be in the form of a slow or time-released particle or capsule such as microparticles, for example, microbeads or a microgel. In accordance with these embodiments, a microparticle can contain a composition disclosed herein and once the microparticles are introduced to a subject in need of such a composition, the composition can be released upon targeting a specific region and/or upon introduction, in timed intervals or as the microparticles degrade. These methods are known in the art and are contemplated herein.

Therapeutic agents may be formulated within a mixture to include about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition.

In another embodiment, nasal solutions or sprays, aerosols or inhalants may be used to deliver the compound of interest. Additional formulations that are suitable for other modes of administration include suppositories and pessaries. A rectal pessary or suppository may also be used. In general, for suppositories, traditional binders and carriers may include, for example, polyalkylene glycols or triglycerides; such suppositories may be formed from mixtures containing the active ingredient in the range of 0.5% to 10%, preferably 1% 2%.

Oral formulations include such normally employed excipients as, for example, pharmaceutical grades of mannitol, lactose, starch, magnesium stearate, sodium saccharine, cellulose, magnesium carbonate and the like. In certain embodiments, oral pharmaceutical compositions can include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations can contain at least 0.1% of active compound.

A pharmaceutical composition may be prepared with carriers that protect active ingredients against rapid elimination from the body, such as time-release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others are known.

### Kits

In certain embodiments, a kit contemplated herein may include compositions for treating a subject having early onset Type 1 diabetes or Type 2 diabetes having remaining insulin-producing islet cells. In certain embodiments, kits may include a short-duration supply of AAT for treatment of a subject having these conditions or a more long term supply as evaluated by a health professional.

### Other Embodiments

Some embodiments include methods for treating a subject including, identifying a subject having Type 1 diabetes; identifying the subjects having Type 1 diabetes that produce insulin; and administering a therapeutically effective amount of a composition comprising alpha-1 antitrypsin (AAT) to the subject, wherein the composition modulates beta cell destruction in the subject. In accordance with these embodiments, insulin production in the subject can be around 1 pmol/ml to about 10 pmol/ml. The subject can have residual beta islet cell function. The composition can further include with at least one other therapeutic treatment for diabetes (e.g. immunologic type treatment). In addition, a subject may be treated with the composition for 2 years or less. Other subjects may be treated for 10 weeks or less. Treatment of a subject may be bi-weekly or one time weekly or one time daily.

In certain embodiments, a treatment may be a commercial source of AAT. Some embodiments may include a treatment of about 10 mg/kg to about 100 mg/kg of AAT to the subject.

Some embodiments report methods for treating a subject including identifying a subject having Type 1 diabetes; identifying the subjects having stimulated c-peptide of ≧0.2 pmol/mL; and administering a therapeutically effective amount of a composition comprising alpha-1 antitrypsin (AAT) to the subject, wherein the composition modulates beta cell destruction in the subject. The composition can be naturally occurring AAT or substantially purified from a wild type, mutant, or transgenic mammalian source or isolated from a culture producing wild type, mutant, or transformed cells. Embodiments concerning treatments of a subject may include delaying loss of c-peptide in the subject compared to an untreated control population.

### EXAMPLES

Examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

### Example 1

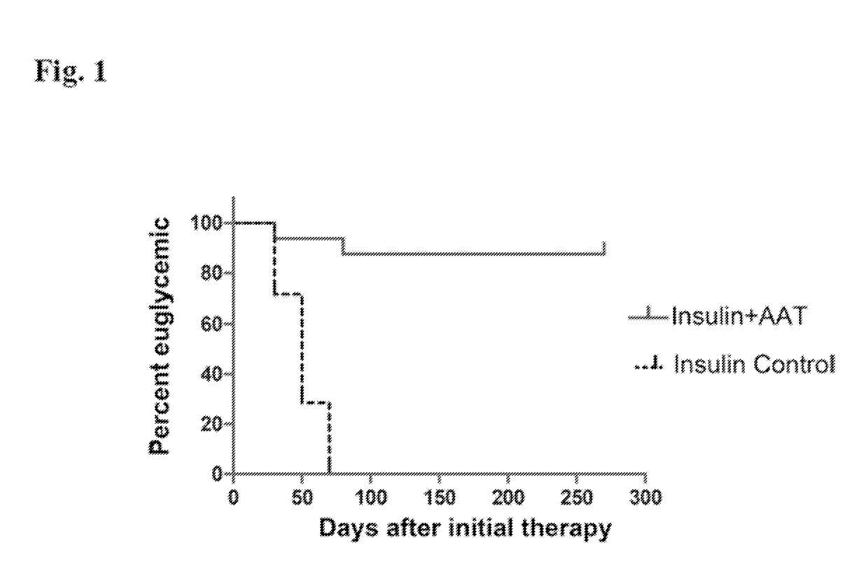

In one previous study, diabetic NOD mice with blood glucoses between 300 and 450 mg/dl were treated with a short 2 week course of human AAT. 88% (21/24) of mice remained euglycemic for >270 days compared to the controls that were treated with insulin therapy (FIG. 2). FIG. 1 illustrates and example of a short term AAT treatment of diabetic NOD mice restores euglycemia. AAT treated mice were compared with insulin control mice by using Wilcoxon signed rank test (P<0.0001) (see for example, FIG. 1, Koulmanda et al, PNAS vol. 105, No. 42 pages 16242-16247).

It was also observed that some subjects had decreased inflammatory mediators in the pancreatic lymph nodes and pancreata of AAT treated mice compared to control NOD mice.

### Example 2

Proposed clinical study: Treat with Alpha₁-antitrypsin (AAT) weekly for 8 weeks in subjects with type 1 diabetes (selected by enclosed criteria) and detectable levels of c-peptide:

Goal of Protocol:
1. Be a safe and feasible treatment for people with diabetes (Type 1 or potentially Type 2)
2. Delay loss of c-peptide
3. Sustain metabolic control of glucose levels as measured by HbA1c and glucose fluctuations

Secondary Outcomes:

How AAT treatment affects the following will be investigated:
1. The area under the c-peptide curve (AUC) of the 2 hour MMTT conducted at the month 12 visit. Levels of c-peptide will be assessed.
2. HbA1c, glucose and insulin changes over time will be analyzed with respect to AAT treatment.
3. Autoantibody levels will be measured to see if these levels are affected under the treatment.
4. AAT levels in the blood will be analyzed.
5. Inflammatory markers (serum cytokines and whole blood cytokine production)
6. Cytokines produced by monocytic stimulated cell populations (e.g. IL-6, IL-18)
7. Elispot assays or other assay known in the art will be used to assess: Interferon Gamma and Interleukin 10 levels before and after treatment
8. Number and severity of adverse events, hypoglycemic events and DKA events

In certain embodiments, the clinical trial will be used to assess participant safety in protocol, feasibility of treatment, assessment of the effects of AAT on the maintenance or stabilizing C-peptide production, to assess the effects of AAT on glycemic variability and HbA1c. In other exemplary methods, underlying mechanisms for how AAT may affect T1D progression will be investigated. In other embodiments, safety and tolerability of AAT in subjects with T1D at a dose of 80 mg/kg will be assessed.

Description of a Population to be Enrolled in a Diabetes Clinical Trial: Study Design and Research Methods

Certain Criteria for Eligibility of some Treatment Regimens:

Cohorts of 10 subjects who are all within 5 years of diagnosis with T1D and continue to produce insulin will be enrolled. It was previously demonstrated that 20% of patients studied, who were within 5 years of diagnosis, had some remaining insulin production (e.g. 2-5 pmol/ml). In some exemplary embodiments, participants selected have a stimulated c-peptide of ≧0.2 pmol/mL. One of the goals in this study is for one clinical outcome to be preserving remaining insulin production while preserving or stimulating islet cell growth and/or production.

The investigators will evaluate safety and efficacy between each cohort of 10 subjects. The enrolled population will still have residual beta cell function and will be producing some of their own insulin. This will enable testing a hypothesis that AAT can slow the loss of c-peptide production in stressed islet cell population as well as more healthy populations.

A. Inclusion Criteria:
1. Diagnosis of Type 1 Diabetes Mellitus based on ADA Criteria for fewer than 5 years but more than 100 days post diagnosis or onset.
2. For this study, the population will be 6-46 years of age, inclusive. To assess safety, 3 patients over the age of 16 will be enrolled. The first 3 patients will be staggered by 2 weeks. There was no stopping criteria for these 3 patients so the age criteria went down to 6 years old.
3. C-peptide increase during screening mixed meal tolerance test with a minimal stimulated value of ≧0.2 pmol/mL
4. Positive for antibodies to insulin (if insulin auto antibody positive only, determination must be within two weeks of insulin initiation), GAD-65, IA-2 or ZnT8
5. Subject must agree to intensive management of diabetes with an HgbA1c goal of <7.0%
6. Serum creatinine ≦1.5× upper limit of normal
7. AST <2 times the upper limit of normal
8. Hematology: WBC ≧3000×10⁹/L; platelets ≧100×10⁹/L; hemoglobin ≧10.0 g/dL.

B. Exclusion Criteria:
1. Unable or unwilling to comply with the requirements of the study protocol
2. Body Mass Index (BMI) >30 kg/m2
3. Unstable blood sugar control defined as one or more episodes of severe hypoglycemia (defined as hypoglycemia that required the assistance of another person) within the last 30 days
4. Previous immunotherapy for T1D
5. Administration of an experimental agent for T1D at any time or use of an experimental device for T1D within 30 days of screening, unless approved by the study PI
6. History of any organ transplant, including islet cell transplant.
7. Active autoimmune or immune deficiency disorder (e.g. sarcoidosis, rheumatoid arthritis)
8. Serum bilirubin>ULN, except those Subjects whose abnormal values were attributed to any stable, benign condition (such as Gilbert's Syndrome) may be included
9. Individuals with a history of IgA deficiency
10. Individuals with a history of hypersensitivity to AAT
11. TSH outside the normal range at screening, except those Subjects on stable doses of thyroid hormone replacement therapy may be included
12. Known HIV positivity, active hepatitis B or active hepatitis C infection
13. Anticipated pregnancy during active dosing or within 3 months after completion of active dosing phase
14. History of a malignant neoplasm within the previous 5 years (except in situ cervical cancer and curable non-melanoma skin malignancy)
15. Any social condition or medical condition that would, in the opinion of the investigator, prevent complete participation in the study or that would pose a significant hazard to the subjects' participation
16. History of active substance abuse within 12 months of screening
17. A psychiatric or medical disorder that would prevent giving informed consent

### AAT Treatment:

Eligible subjects will be treated once a week for 8 weeks (8 total treatments). The subjects will then be followed for safety and efficacy every 3 months for the following year and every 6 months for the second year. AAT will be provided by Baxter pharmaceuticals. AAT at 80 mg/kg will be infused at a rate of 0.08 ml/kg body weight per minute. A 50 kg subject would receive 4000 mg AAT. A 1000 mg vial of AAT is diluted in 50 mls. The dose of 4000 mg dilutes to 200 mls. At a rate of 0.08 ml/kg/min the subject will receive 4 ml/min. A 200 ml infusion at 4 ml/min would take 50 min. Typically, these infusions will last under 1 hour. Infusions will take place at the outpatient CTRC. All subjects will receive study drug, this is an open label trial.

Blood draws can be used to assess CBC, Chemistry, HbA1c, T cells, AAT level, D-dimers, c-peptide, PT/PTT and other agents found in the blood.

Participants will not be pretreated with any medication; however acetaminophen and diphenhydramine will be available at the bedside for treatment of any reactions. Vitals will be taken prior to AAT administration and for every 30 minutes for the duration of the infusion. CBC, chemistry and urine pregnancy (if female) tests will be done and reviewed prior to each infusion. Patients will be contacted daily for 2 days following each infusion to assess any adverse events.

### Intensive Diabetes Management

All subjects enrolled in this study will undergo intensive management of their diabetes. One goal of management will be HbA1c levels within the currently recommended American Diabetes Association age specific target range in the absence of significant hypoglycemia or diabetic ketoacidosis. Subjects will be expected to take a sufficient number of daily insulin shots to meet this goal. Alternatively, subjects may use insulin pump therapy. Subjects will also be expected to check blood sugar levels at least 4 times a day. A Diabetes Management Team will be working with the subjects to achieve these goals. A member of the diabetes management team may contact the subject's primary diabetes treating physician about possible adjustments in insulin regimen, referral to a dietician, or other approaches that would improve glucose control if necessary. Participants who fail to achieve glucose control within the ADA goals will not be excluded from the study, but additional measures will be instituted to improve glucose control.

### Continuous Glucose Management

A continuous glucose monitoring (CGM) device will be used to measure more accurately hyperglycemic and hypoglycemic episodes and to examine the effect of AAT on the variability of blood glucose levels. CGM will be performed for a target of seven days at each designated time point. The exact time will depend upon the time the subcutaneous sensor remains inserted. The study's CGM monitoring outputs will be blinded to the subjects.

CGM outputs will be reviewed for adequacy of information capture; in the event of an inadequate tracing, the CGM session will be repeated. The screening CGM measurement should be obtained prior to study drug dosing.

### MMTT (Mixed Meal Tolerance Tests)

This procedure will be done during screening and at months 3, 6, 12, 18, and 24. This test monitors how much insulin the beta cells are making and is a valuable test for assessing progress of subjects described herein before, during and after AAT treatments. Participants will be given specific instructions on insulin dosing and diet for 3 days prior to the MMTT. On the morning of the test, participants may only drink water and will take no short acting insulin within 2 hours of the test. An IV will be placed and blood will be drawn from it. The participant will drink “Boost High Protein Nutritional Energy Drink” in 5 minutes. Blood will be drawn from the IV 11 times over the next 4 hours. The amount of blood taken for this test will be about 3 tablespoons (17 ml). This is in addition to the volume of blood taken for the other tests at the visits. Blood sugar will be adjusted as necessary after these samples are taken.

Multiple forms and multiple commercial products of AAT exist. The instant study is using ARALAST-NP™ but other commercially available AAT compositions exist (e.g. ProlastinC™, Kamata's aerosol version etc). In addition, AAT can be readily purified from blood plasma or caboxyterminal peptides can be prepared and used that have AAT activities contemplated herein.

ARALAST-NP™ is an AAT that is currently FDA approved (2002) for the treatment of hereditary emphysema and manufactured by Grifols Biologicals Inc for Baxter Healthcare Corporation. The safety of ARALAST-NP™ has been established in preclinical studies, clinical studies as well as post marketing practice. Overall, 1.3% of treated patients experience infusion issues related to ARALAST-NP™. One Phase 3 double-blinded cross-over study examined ARALAST-NP™ in patients with Alpha1-Proteinase Inhibitor Deficiency. A total of 28 subjects were randomized and received 60 mg/kg ARALAST-NP™ once a week for 10 weeks. No serious adverse events were reported during the 10 weeks of treatment of the subsequent 13 weeks of follow-up. As of 2005, there were 426 patients treated with ARALAST-NP™ (over 12,000 infusions). 93 of these patients have received ARALAST-NP™ for over 14 months. A total of 28 adverse events have been reported out of the 426 patients treated and none of these were considered serious. No adverse event related changes have been made to the ARALAST-NP™ package between 2002 and 2005. There have been no reports past 2005. The investigational brochure does not mention the proximity of the adverse events to the infusion of the medication, but does mention that no serious adverse events have been reported as related to ARALAST-AP treatment.

In the pediatric population, intravenous AAT has been studied in 106 preterm infants (placebo and AAT treatment) in an attempt to prevent chronic lung disease of prematurity. Doses given were 120 mg/kg each week for two weeks. During the study, infusions were well tolerated and the complications of prematurity were not different between the groups except for pulmonary hemorrhage (9 in the placebo group and 2 in the AAT group). The surviving children were assessed-at >18 months of age and there was no difference in risk of severe neurodevelopmental abnormality (frequency of cerebral palsy and/or mental retardation, legal blindness and/or deafness).

Clinical secondary endpoints will examine changes in HbA1c, glucose changes, insulin changes, autoantibodies, c-peptide and hypoglycemic events (defined as those that required assistance). Immunologic secondary endpoints examine AAT levels, inflammatory markers, cytokines produced by simulated monocytic cell populations and whole blood, and ELISPOT. One repeated measure ANOVA will be used to analyze continuous variables and non-linear mixed effects model (logistic or Poisson regression, as appropriate) will be used to analyze counting or binary variables.

In order to describe efficacy, the treatment group will be compared to the results obtained in a previous TrialNet MMF/DZB study. The MMF/DZB study had a placebo controlled group that also received intensive diabetes management. The results from this previous study will be used to compare to the treated population in the currently proposed study.

The study is an intent-to-treat analysis. Therefore, all enrolled participants will be included in the analysis of both primary and secondary endpoints regardless of duration of treatment received.

Good diabetes control can reduce the rate of developing diabetes related long-term complications. Interventions, such as AAT, can help maintain healthy islet cells and some insulin production in a diabetic population. Even minimal insulin production has been related to better metabolic control and less long-term complications. Currently, the majority of intervention therapies for T1D use immunosuppressant treatment. These treatments are not without risk. If they are found to be efficacious, anti-inflammatory treatment, such as AAT, would propose less risk to side effects and more long term benefit to a subject having T1D with residual islet cell activities.

TABLE 1

| | Blood | | Treatment Period | | | | | | | | | | | | | Follow-up Visits | |
|---|---|---|---|---|---|---|---|---|---|---|---|---|---|---|---|---|---|
| | volume | Screen | (Weeks) | | | | | | | | | | | | | (Months) | |
| Week | MLS | −6 | 0 | 1 | 2 | 3 | 4 | 5 | 6 | 7 | 9 | Mo 3 | Mo 6 | Mo 9 | Mo 12 | Mo 18 | Mo 24 |
| Eligibility Activities | | X | | | | | | | | | | | | | | | |
| Drug administration | | | X | X | X | X | X | X | X | X | | | | | | | |
| Vital Signs | | X | X | X | X | X | X | X | X | X | X | X | X | X | X | X | X |
| Exam & history | | X | X | X | X | X | X | X | X | X | X | X | X | | X | | X |
| CMP (Chem) | 5 | X | X | X | X | | X | | | X | X | X | X | X | X | X | X |
| CBC | 2 | X | X | X | X | X | X | X | X | X | X | X | X | X | X | X | X |
| D-dimmers, PT INR, | 3 | | X | | | | X | | | | | X | | | | | |
| PTT | | | | | | | | | | | | | | | | | |
| HgbA1c | 0.2 | X | X | | | | | | | | | X | X | X | X | X | X |
| T1D antibodies | 3 | X | | | | | | | | | | X | X | | X | X | X |
| ELISPOT | 50 | | X | | | | | | | X | | X | X | | X | X | X |
| HLA Typing | 3 | | X | | | | | | | | | | | | | | |
| Pregnancy test | | X | X | X | X | X | X | X | X | X | X | X | X | | | | |
| Hep B and C and IgA | 5 | X | | | | | | | | | | | | | | | |
| EBV, CMV serologies | 4 | X | | | | | | | | | | X | | | | | |
| Flow (Zipris) | 10 | x | | X | | X | | | | X | X | X | X | X | X | X | X |
| Cytokines (Zipris) | 2 | X | | X | | X | | | | X | X | X | X | X | X | X | X |
| Whole blood | 3 | X | | X | | X | | | | X | X | X | X | | X | X | X |
| cytokines (Shapiro) | | | | | | | | | | | | | | | | | |
| MMTT | 17 | X | | | | | | | | | | X | X | | X | X | X |
| CGM | | X | | | | | | | | | | | X | | X | | X |
| Review SMBG [symbol] | | X | X | X | X | X | X | X | X | X | X | X | X | X | X | X | X |
| Insulin requirements | | X | X | X | X | X | X | X | X | X | X | X | X | X | X | X | X |
| Con. Meds & AEs | | X | X | X | X | X | X | X | X | X | X | X | X | X | X | X | X |
| Blood Volume By | | 52 | 64 | 22 | 7 | 22 | 10 | 7 | 7 | 72 | 19 | 96 | 89 | 16 | 89 | 89 | 89 |
| visit (MLS) | | | | | | | | | | | | | | | | | |
| Blood Volume by 6 | | 116 | | | | | | | | | | | | | | | |
| Week segments | | | 129 | | | | | | | | | | | | | | |
| (MLS) | | | | 75 | | | | | | | | | | | | | |
| | | | | | 125 | | | | | | | | | | | | |
| | | | | | | 115 | | | | | | | | | | | |
| | | | | | | | | | | | 115 | | | | | | |
| Week | | −6 | 0 | 1 | 2 | 3 | 4 | 5 | 6 | 7 | 8 | 9 | Mo 3 | Mo 6 | Mo 12 | Mo 18 | Mo 24 |

[symbol]

indicates data missing or illegible when filed

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating a subject comprising,
identifying a subject having Type 1 diabetes;
identifying the subjects having Type 1 diabetes that can produce insulin; and
administering a therapeutically effective amount of a composition comprising alpha-1 antitrypsin (AAT) or carboxyterminal peptide derived from AAT to the subject, wherein the composition modulates beta cell destruction in the subject.

2. The method of claim 1, wherein insulin production in the subject is around 1 pmol/ml to about 10 pmol/ml.

3. The method of claim 1, wherein the subject has residual beta islet cell function.

4. The method of claim 1, further comprising combining the composition with at least one other therapeutic treatment for diabetes.

5. The method of claim 1, wherein the subject is treated with the composition for 2 years or less.

6. The method of claim 1, wherein the subject is treated with the composition for 10 weeks or less.

7. The method of claim 1, wherein treatment of the subject comprises a treatment that is bi-weekly or one time weekly or one time daily.

8. The method of claim 1, wherein the treatment comprises a commercially available source of AAT.

9. The method of claim 1, wherein the treatment comprises about 20 mg/kg to about 100 mg/kg of AAT to the subject per treatment.

10. The method of claim 1, wherein the treatment comprises administering a composition having a carboxyterminal peptide derived from AAT.

11. A method for treating a subject comprising,
identifying a subject having Type 1 diabetes;
identifying the subjects having stimulated c-peptide of ≧0.2 pmol/mL; and
administering a therapeutically effective amount of a composition comprising alpha-1 antitrypsin (AAT) or carboxyterminal peptide derived from AAT to the subject, wherein the composition modulates beta cell destruction in the subject.

12. The method of claim 11, wherein the composition comprises naturally occurring AAT.

13. The method of claim 11, wherein the AAT is substantially purified from a wild type, mutant, or transgenic mammalian source.

14. The method of claim 11, wherein the AAT is isolated from a culture producing wild type, mutant, or transformed cells.

15. The method of claim 11, wherein AAT or the carboxyterminal peptide derived from AAT dose ranges from about 1 mg/kg to about 100 mg/kg of body weight of the subject.

16. The method of claim 11, wherein the composition is administered parenterally, orally, nasally, buccally, intravenously, intramuscularly, subcutaneously, intrathecally, transdermally, by osmotic pump, by inhalation, or a combination thereof.

17. The method of claim 11, wherein the treatment delays the loss of or stabilizes c-peptide production in the subject.

18. The method of claim 11, wherein the composition is administered at least once weekly or once daily.

19. The method of claim 11, wherein the subject has Type 1 diabetes and has detectible c-peptide levels.

20. The method of claim 11, wherein the subject is a juvenile.

21. The method of claim 11, wherein the treatment comprises 40 mg/kg to about 100 mg/kg of AAT or carboxyterminal peptide of AAT to the subject per treatment.

22. The method of claim 11, wherein the use of insulin in the subject is reduced compared to a control not receiving the composition.
